(19) United States
(12) Patent Application Publication
(10) Pub. No.: US 2019/0056122 A1
(43) Pub. Date: Feb. 21, 2019

(54) Clean Rooms Having Dilute Hydrogen Peroxide (DHP) Gas and Methods of Use Thereof

(71) Applicant: Synexis LLC, Kansas City, MO (US)

(72) Inventors: James D. LEE, Kansas City, MO (US); Douglas J. BOSMA, Kansas City, MO (US)

(73) Assignee: Synexis LLC, Kansas City, MO (US)

(21) Appl. No.: 15/567,564

(22) PCT Filed: Apr. 20, 2016
(86) PCT No.: PCT/US16/28457

§ 371 Date: Oct. 18, 2017

Related U.S. Application Data
(60) Provisional application No. 62/149,925, filed on Apr. 20, 2015.

(51) Int. Cl. F24F 3/16; A61L 2/20; B01L 1/04

(52) U.S. Cl. CPC F24F 3/161; B01L 1/04; A61L 2/208

(57) ABSTRACT

Provided are improved clean rooms having dilute hydrogen peroxide (DHP) gas that provides for antiseptic conditions. Also provided are DHP gas containing clean rooms that have reduced levels of volatile organic compounds (VOCs) and methods for preparing clean rooms having DHP gas.

### CROSS-REFERENCE TO RELATED APPLICATIONS

This application is a U.S. National Phase application of International Application No. PCT/US2016/028457 filed Apr. 20, 2016, which is related to U.S. Provisional Patent Application No. 62/149,925 filed Apr. 20, 2015, each of which are incorporated herein in their entireties.

### FIELD OF THE INVENTION

This present disclosure relates generally to improved clean rooms having dilute hydrogen peroxide (DHP) gas that provides for antiseptic conditions. The present disclosure further relates to DHP gas containing clean rooms that have reduced levels of volatile organic compounds (VOCs). The present disclosure also relates to methods of preparing clean rooms having DHP gas.

### BACKGROUND OF THE INVENTION

Hydrogen peroxide (H₂O₂) is a strong oxidant and has well known antimicrobial and antiseptic properties as well as activity against organic compounds. H₂O₂ also has activity against volatile organic compounds (VOCs) oxidizing them and hydrolyzing them and breaking them down. Hydrogen peroxide hydrolyzes, among other things, formaldehyde, carbon disulfide, carbohydrates, organophosphorus and nitrogen compounds, and many other more complex organic molecules. H₂O₂ is produced commercially in large quantities as either a colorless liquid or as an aqueous solution, generally from about 3 to 90%. See, Merck Index, 10^{th} Edition at 4705 to 4707. It has recently been shown that H₂O₂ can be produced as a purified hydrogen peroxide gas (PHPG) that is free of ozone, plasma species, or organic species.

PHPG is a non-hydrated gaseous form of H₂O₂ that is distinct from liquid forms hydrogen peroxide including hydrated aerosols and vaporized forms. Aerosolized and vaporized forms of hydrogen peroxide solution have significantly higher concentrations of H₂O₂, typically comprising greater than 1×10⁶ molecules per cubic micron compared to air containing PHPG that contains between 5 and 25 molecules per cubic micron. Hydrogen peroxide aerosols and vapors are prepared from aqueous solutions of hydrogen peroxide and also differ from PHPG as the aerosols are hydrated and, regardless of the size of the droplet, settle under the force of gravity. Vaporized forms condense and settle. Aerosolized forms of hydrogen peroxide are effective antimicrobial agents however they are generally considered toxic and wholly unsuitable for use in occupied spaces. See for example, Kahnert et al., “Decontamination with vaporized hydrogen peroxide is effective against Mycobacterium tuberculosis,” Lett Appl Microbiol. 40(6):448-52 (2005). The application of vaporized hydrogen peroxide has been limited by concerns of explosive vapors, hazardous reactions, corrosivity and worker safety. See Agalloco et al., “Overcoming Limitations of Vaporized Hydrogen Peroxide,” Pharmaceutical Technology, 37(9):1-7 (2013). Further, spaces treated with aerosolized forms, typically at concentrations of between 150 to 700 ppm, remain unsuitable for occupation until the H₂O₂ has been reduced by degradation to water and oxygen and the H₂O₂. The use of PHPG solves the problem of toxicity of aerosolized H₂O₂. Vaporized and liquid forms of H₂O₂ and can provide continuous safe antimicrobial and oxidative activity.

PHPG is non-hydrated and behaves essentially as an ideal gas. In this form PHPG behaves largely as an ideal gas, capable of diffusing freely throughout an environment to attain an average concentration of about 25 molecules per cubic micron of air. As a gas, PHPG is capable of penetrating most porous materials essentially diffusing freely to occupy any space that is not air tight. The gaseous form of hydrogen peroxide doesn't settle, deposit, or condense when present at concentrations up to 10 ppm. PHPG is completely “green” and leaves no residue as it breaks down the water and oxygen.

Importantly, and in contrast to vaporized and aerosolized forms of H₂O₂, environments containing up to 1 ppm H₂O₂ have been designated as safe for continuous human occupation under current Occupational Safety and Health Administration (OSHA), National Institute for Occupational Safety and Health (NIOSH), or American Conference of Industrial Hygienists (ACGIH) standards. It is believed that 10 ppm is also safe for human occupation though not yet recognized by the regulatory authorities. With the advent of PHPG generating devices, appropriate studies can now be performed. The ability to produce effective amounts of PHPG, the safety of PHPG when present as a dilute hydrogen peroxide (DHP) gas combined with its effectiveness as an antimicrobial agent provides a myriad of useful applications.

U.S. Pat. No. 8,168,122 issued May 1, 2012 and U.S. Pat. No. 8,685,329 issued Apr. 1, 2014, both to Lee disclose methods and devices to prepare PHPG for microbial control and/or disinfection/remediation of an environment. International Patent Application No. PCT/US2015/029276, published as International Patent Publication No. WO 2015/171633 provides improved PHPG generation methods and devices capable of achieving higher steady state levels of PHPG. International Patent Application No. PCT/US2014/038652, published as International Patent Publication No. WO 2014/186805 discloses the effectiveness and use of PHPG for the control of arthropods, including insects and arachnids. International Patent Application No. PCT/US2014/051914, published as Feb. 26, 2015 as International Patent Publication No. WO/2015/026958 discloses the beneficial effects of PHPG on respiratory health, including increased resistance to infection and increased hypothiocyanate ion in mammalian lungs. The contents of each of the foregoing applications are incorporated herein by reference in their entireties.

Since their introduction in the 60's, clean rooms have become increasingly important in both the industrial and health care settings. In addition to their extensive use in semiconductor manufacturing, clean rooms are used in the production of pharmaceuticals as well as in biomedical research facilities, for example as part of biosafety environments. Clean rooms provide for the control and reduction of airborne particles such as dust using filtration methods and are characterized by the size, number and distribution of airborne particles. Clean rooms are generally not maintained as sterile environments, though UV lights may be used in a limited way to reduce microorganism loads. Clean rooms can also be equipped with ventilation systems to remove volatile compounds, though only compounds and particles that are airborne can be removed.

Clean rooms are classified according to the number and size of particles permitted per volume of air. The classification and standards for clean rooms have been established by the International Organization for Standardization (ISO). ISO 14644 standards were initially documented under U.S. Federal Standard 209E (FS 209E). The current version of the standard is ISO 14644-2 that published in 2000. These standards and methods to achieve the standards are known in the art.

Related to clean rooms are enclosed laboratory facilities that provide various levels of containment so that potentially dangerous biological agents are not released and to protect workers from potential contamination. There are four levels, BSL-1 to BSL-4, that are specified in the U.S. by the Centers for Disease Control (CDC). See http://www.cdc.gov/biosafety/publications/bmb15/BMBL5_sect_IV.pdf. Federal guidelines for Biosafety in Microbiological and Biomedical Laboratories (BMBL) are known to one of skill in the art and the most recent version is BMBL, 5^{th} edition (December 2009). The BMBL can be found on the internet at www.cdc.gov/biosafety/publications/bmb15/. Similar levels are defined in the European Union and elsewhere.

The materials used in the manufacture of clean rooms are selected to eliminate the production of particles. Thus, even common materials such as paper and natural fiber materials are excluded from clean rooms as these can be significant sources of particle contamination. Therefore, clean rooms are constructed of hard impervious materials with a smooth finish and sharp angles and edges are reduced to prevent particle formation. Among the suitable materials are phenolic plastics, glass reinforced plastics, and steel. Where more common materials such as drywall are used, there is a need to seal and finish the surface to prevent the production of particles. Notably, many of the materials outgas unwanted organic species that can interfere with the purposes of the clean room. More specifically, various organic species derived from the clean room construction materials themselves can create impurities on the surface of silicon wafers during semiconductor production. Table 1 below provides examples of compounds outgassed from common clean room construction materials. These organic compounds are undesirable.

TABLE 1
Clean room Construction Materials and Their Outgassing Compounds

| Construction Material | Organic Compounds Outgassed |
|---|---|
| Flooring materials | Dioctyl phthalate (DOP) |
| HEPA gel seal | Triethylphosphate (TEP) |
| Urethane from sealants | TEP and butylated hydroxytoluene (BHT) |
| for HVAC | |
| Polyurethane adhesives | BHT, amine compounds |
| Flexible duct connector | Phosphate esters, DOP |
| Concrete sealing paint | Alkenes, alcohols, amines |
| Silicon sealant | Cyclic siloxanes |
| Vinyl material | DOP, texanol isobutyrate (TXIB), tributyl |
| | phosphate (TBP) |
| Silicon tubing | Siloxanes, dibutyl phosphate (DBP) |

Source: Gutowski, T., Oikawa, H., and Kobayashi, S. airborne Molecular contamination Control of Materials Utilized in the Construction of a Semiconductor Manufacturing Facility. 1997 SPWCC Proceedings, vol. II, page 143.

There is a need for clean rooms that can eliminate or destroy compounds that are given off by the materials used to manufacture clean rooms as these compounds, for example, interfere in the production of semiconductors.

Further, improved clean rooms that can remove or destroy unwanted organic compounds, for example organic compounds that settle onto surfaces in the clean room, is desirable. Improved clean rooms providing for the destruction of organic compounds in the clean room environment and prior to filtration and other removal methods is also highly desirable.

Clean rooms are generally directed to the elimination of particles and are not sterile. Therefore, improved clean room facilities that provide for the reduction or elimination of microorganisms such as bacteria, fungi, molds, and viruses are desirable.

### SUMMARY OF THE INVENTION

The present disclosure provides for, and includes, clean rooms comprising Dilute Hydrogen Peroxide (DHP) gas at a concentration of at least 0.05 parts per million.

More particularly, the present disclosure provides for an ISO 14644 class 1 clean room having at least 0.05 ppm DHP gas, an ISO 14644 class 2 clean room, an ISO 14644 class 3 clean room, an ISO 14644 class 4 clean room, an ISO 14644 class 5 clean room, an ISO 14644 class 6 clean room, an ISO 14644 class 7 clean room, or an ISO 14644 class 8 clean room comprising Dilute Hydrogen Peroxide (DHP) gas at a concentration of at least 0.05 parts per million.

The present disclosure provides for and includes, methods to prepare clean rooms comprising Dilute Hydrogen Peroxide (DHP) gas at a concentration of at least 0.05 parts per million comprising providing one or more PHPG producing devices to an clean room.

The present disclosure provides for and includes, methods to prevent contamination of a clean room by microorganisms comprising providing a Dilute Hydrogen Peroxide (DHP) gas at a concentration of at least 0.05 parts per million (ppm) to a clean room.

The present disclosure provides for and includes, methods for reducing contamination of a clean room by microorganisms comprising providing a Dilute Hydrogen Peroxide (DHP) gas at a concentration of at least 0.05 parts per million (ppm) to a clean room.

The present disclosure provides for and includes, methods for eliminating contamination of a clean room by microorganisms comprising providing a Dilute Hydrogen Peroxide (DHP) gas at a concentration of at least 0.05 parts per million (ppm) to a clean room.

The present disclosure provides for and includes, methods for reducing organic compounds in a clean room comprising providing a Dilute Hydrogen Peroxide (DHP) gas at a concentration of at least 0.05 parts per million (ppm) to a clean room.

### DETAILED DESCRIPTION

Unless defined otherwise, technical and scientific terms as used herein have the same meaning as commonly understood by one of ordinary skill in the art. One skilled in the art will recognize many methods can be used in the practice of the present disclosure. Indeed, the present disclosure is in no way limited to the methods and materials described. Any references cited herein are incorporated by reference in their entireties. For purposes of the present disclosure, the following terms are defined below.

As used herein, Purified Hydrogen Peroxide Gas (PHPG) and Dilute Hydrogen Peroxide (DHP) gas are used interchangeably. Purified hydrogen peroxide gas as used herein is non-hydrated, substantially free of ozone, plasma species, and organic species. Also as used herein, the level of PHPG in a room is determined as the steady state level of PHPG in a clean room. Clean rooms according to the present disclosure comprising DHP gas are clean rooms having a steady state concentration of DHP gas of at least 0.05 ppm for a period of at least 15 minutes. Notably, during normal use, PHPG is used up as it reacts with organic compounds, reacts with microorganisms, or otherwise degrades and thus must be continually replaced. In practice, it is anticipated that the clean rooms according to the present disclosure, are maintained in a DHP gas containing state by the constant production of PHPG via one or more devices as part of the heating ventilation and air conditioning (HVAC) system or supplied by one or more stand alone PHPG producing devices.

As used herein, the singular form “a,” “an” and “the” includes plural references unless the context clearly dictates otherwise. For example, the term “a bacterium” or “at least one bacterium” may include a plurality of bacteria, including mixtures thereof. In another example, the term “a fungi” or “at least one fungi” may include a plurality of bacteria, including mixtures thereof. Similarly, “a VOC” or “at least one VOC” may include multiple VOCs and mixtures thereof.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “higher” refers to at least about 3%, 5%, 7%, 10%, 15%, 20%, 25%, 30%, 50%, 60%, 70%, 80%, 90%, or even a few folds higher.

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or greater increase.

As used herein the term “less” refers to at least about 3%, 5%, 7%, 10%, 15%, 20%, 25%, 30%, 50%, 60%, 70%, 80%, 90%, or even a few folds higher.

As used herein the term “reducing” or “decreasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or greater increase.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The present disclosure provides for, and includes, a clean room comprising Dilute Hydrogen Peroxide (DHP) gas at a concentration of at least 0.05 parts per million (ppm) that conform to one or more national or international standards, including but not limited to US 209D dated 1988, US 209E dated 1992, UK standard BS 5295 dated 1989, Australian standard AS 1386, French standard AFNOR X44101 dated 1972, German standard VD 1.2083, dated 1990 and ISO standard 14644-1 and 14644-2 dated 2000. Also included and provided for in the present disclosure are any rooms and or areas that are designed to reduce airborne particles and compounds. Clean rooms conforming to that conform to one or more national or international standards include levels of DHP gas as provided in paragraphs [0046] and [0047].

In aspects according to the present disclosure, the clean room conforms to ISO 14644-2 standard. In an aspect, the clean room is an ISO 14644 class 1 clean room comprising DHP gas at a concentration of at least 0.05 parts per million (ppm). In another aspect, the clean room is an ISO 14644 class 2 clean room comprising DHP gas at a concentration of at least 0.05 parts per million (ppm). In another aspect, the clean room is an ISO 14644 class 3 clean room comprising DHP gas at a concentration of at least 0.05 parts per million (ppm). In another aspect, the clean room is an ISO 14644 class 4 clean room comprising DHP gas at a concentration of at least 0.05 parts per million (ppm). In another aspect, the clean room is an ISO 14644 class 5 clean room comprising DHP gas at a concentration of at least 0.05 parts per million (ppm). In another aspect, the clean room is an ISO 14644 class 6 clean room comprising DHP gas at a concentration of at least 0.05 parts per million (ppm). In another aspect, the clean room is an ISO 14644 class 7 clean room comprising DHP gas at a concentration of at least 0.05 parts per million (ppm). In another aspect, the clean room is an ISO 14644 class 8 clean room. As provided herein, clean rooms conforming to the ISO 14644-2 standard include levels of DHP gas as provided in paragraphs [0046] and [0047].

In another aspect, the clean room conforms to British Standard 5295, published in 1989. In an aspect, the clean room is a BS 5295 class 1 clean room comprising DHP gas at a concentration of at least 0.05 parts per million (ppm). In another aspect, the clean room is a BS 5295 class 2 clean room comprising DHP gas at a concentration of at least 0.05 parts per million (ppm). In another aspect, the clean room is a BS 5295 class 3 clean room comprising DHP gas at a concentration of at least 0.05 parts per million (ppm). In another aspect, the clean room is a BS 5295 class 4 clean room comprising DHP gas at a concentration of at least 0.05 parts per million (ppm). As provided herein, clean rooms conforming to British Standard 5295 include levels of DHP gas as provided in paragraphs [0046] and [0047].

The present disclosure also included clean rooms comprising Dilute Hydrogen Peroxide (DHP) gas at a concentration of at least 0.05 parts per million (ppm) that conform to EU GMP Standards. In an aspect the clean room is an EU GMP grade A clean room comprising DHP gas. In another aspect, the clean room is an EU GMP grade B clean room comprising DHP gas. In yet another aspect, the clean room is an EU GMP grade C clean room comprising DHP gas. In another aspect, the clean room is an EU GMP grade D clean room comprising DHP gas at a concentration of at least 0.05 parts per million (ppm). As provided herein, clean rooms conforming to EU GMP Standards include levels of DHP gas as provided in paragraphs [0046] and [0047].

The present disclosure also provides for, and includes, a clean room comprising Dilute Hydrogen Peroxide (DHP) gas at a concentration of at least 0.05 parts per million (ppm) that conforms to EU GMP Standard dated Jan. 1, 1997 and as provided in the Revision of the Annex to the EU Guide to Good Manufacturing Practice-Manufacture of Sterile Medicinal Products.

Clean rooms of the present disclosure may comprise an entire building, one or more rooms within a building, or can be constructed as a modular systems within a larger room. In some aspects, the DHP gas of the clean rooms of the present disclosure can be provided by the building HVAC system, modified with one or more DHP generating devices. In some aspects, a clean room of the present disclosure can comprise a dedicated HVAC system capable of delivering PHPG to the clean room environment.

The present disclosure also provides for modular clean room designs having a separate ventilation system having a dedicated DHP gas generating device. Such clean rooms take the conditioned ambient air and with a second HVAC system, provide a source of DHP gas. Such standalone systems (e.g., a clean room within a room) may further include additional filtering and humidification functions. In some aspects, a modular clean room can provide for isolation of equipment within a facility. Such modular clean rooms may tolerate higher levels of particulate matter than a standard clean room (e.g., ISO 1 to ISO 9) and can be used to isolate critical steps in a production process. In certain aspects a modular clean room may be partially open to the enclosing room. When open to the enclosing room, a modular clean room will generally operate with high flow rates of filtered air such that the flow prevents introduction of unwanted particles and materials. As provided herein, the modular clean room is provided with one or more DHP gas generating devices to maintain a level of DHP gas of at least 0.05 parts per million. Modular clean rooms can be maintained with 0.05 to 10 ppm DHP gas and as provided at paragraphs [0046] and [0047].

Modular clean rooms can be constructed using methods known in the art and supplied with DHP gas to provide sterile environments, environments with reduced contaminants, or both. Numerous manufacturers of modular clean rooms exist, including without limit, Starr Co. (MO), Precision Environments, Inc (OH), PortaFab Corporation (MO), Cambridge Cleanroom Corporation (MA), Modular Cleanrooms Inc. (CO), Terra Universal. Inc. (CA), and American Cleanroom Systems (CA).

At a minimum, modular clean rooms need only provide an enclosed space for the accumulation of DHP gas. Thus, a modular clean room suitable for a DHP gas containing clean room can be a plastic softwall design. Modular clean rooms are not limited by size and can be equipped with multiple DHP gas generating devices to achieve a DHP gas level of between 0.5 ppm and 10 ppm. The use of modular clean rooms, both hard shell and soft wall designs, means that DHP gas containing clean rooms can be developed for individual pieces of equipment in manufacturing process. As provided in Example 2 below, the application of DHP gas containing modular clean room designs to the soft drink bottling process can significantly reduce costs by extending the life of existing equipment. This unexpected improvement suggests that the application of DHP technology to existing systems will yield significant benefits.

The present disclosure provides for and includes, clean rooms, for example as described above that have significantly higher levels of DHP. In certain aspects, the DHP gas level can be up to 10 ppm. In certain aspects, the DHP level ranges between 0.05 and 10 ppm. In one aspect, the concentration of DHP gas in a clean room of the present disclosure is at least 0.08 ppm. In another aspect, the concentration of DHP gas is at least 1.0 ppm. In yet another aspect, the concentration of DHP gas is at least 1.5 ppm. In one aspect, the concentration of DHP gas in a clean room of the present disclosure is at least 2.0 ppm. In another aspect, the concentration of DHP gas is at least 3.0 ppm. In one aspect, the concentration of DHP gas is at least 4.0 ppm. In one aspect, the concentration of DHP gas is at least 5.0 ppm. In another aspect, the concentration of DHP gas in a clean room of the present disclosure is at least 6.0 ppm. In one aspect, the concentration of DHP gas is less than 10 ppm. In one aspect, the concentration of DHP gas is less than 9.0 ppm. In another aspect, the concentration of DHP gas is less than 8.0 ppm. In an aspect, the concentration of DHP gas is less than 7.0 ppm. In another aspect, the concentration of DHP gas is between 0.05 ppm and 10.0 ppm. In yet another aspect, the concentration of DHP gas is between 0.05 ppm and 5.0 ppm. In one aspect, the concentration of DHP gas is between 0.08 ppm and 2.0 ppm. In yet another aspect, the concentration of DHP gas is between 1.0 ppm and 3.0 ppm. In one aspect, the concentration of DHP gas in a clean room of the present disclosure is between 1.0 ppm and 8.0 ppm, or between 5.0 ppm and 10.0 ppm. In other aspects, the concentration of DHP in a clean room cycles between higher and lower concentrations of DHP. By way of non-limiting example, the DHP may be maintained at a higher concentration during the overnight hours and a lower concentration during the daytime hours.

In some aspects, the final concentration of DHP depends on whether the enclosed environment is occupied by a human. Current safe limits for continuous exposure to DHP has been established by the Occupational Safety and Health Administration (OSHA), the National Institute of Occupational Safety and Health (NIOSH), or the Environmental Protection Agency (EPA) to not exceed 1.0 ppm. Accordingly, in certain aspects, the concentration of DHP in a clean room to be occupied by a human does not exceed 1.0 ppm. In another aspect, the concentration of DHP in a clean room occupied by a human does not exceed 0.6 ppm. In another aspect, the concentration of DHP in a clean room occupied by a human does not exceed 0.4 ppm. In another aspect, the concentration of DHP in a clean room occupied by a human does not exceed 0.2 ppm, or does not exceed 0.10 ppm. In one aspect, the concentration of DHP in a clean room occupied by a human does not exceed the limits established by the Occupational Safety and Health Administration (OSHA), the National Institute of Occupational Safety and Health (NIOSH), or the Environmental Protection Agency (EPA).

It has been noted that the mammalian lung itself has levels of hydrogen peroxide that considerably exceeds the OSHA standards and the levels of DHP gas as provided in the present disclosure. Specifically, the moist surfaces of a human lung comprise up to 60,000 molecules per cubic micron (e.g., 1.8 ppm) and hydrogen peroxide is exhaled in every breath. In contrast, DHP gas, at 1 ppm comprises only 25 molecules of H₂O₂ per cubic micron of air. Accordingly, it is believed that levels of 10 ppm or more will be deemed safe for continuous human occupation. The present disclosure provides for, and includes, clean rooms for use and occupation by people that have higher levels of DHP gas, including DHP gas up to 10 ppm. In certain aspects, if necessary should standards not change, the people may be provided with filters or apparatus to eliminate respiration of DHP gas or limited by the amount of time spent exposed to higher levels of DHP gas. Notably, DHP gas quickly dissipates if not replenished. It has been observed that an environment comprising 0.6 ppm DHP gas reverts to undetectable levels within about 15 minutes.

The present disclosure also provides for, and includes, a clean room having DHP gas provided by the heating ventilation and air conditioning (HVAC) system. In certain aspects, the HVAC includes one or more PHPG producing devices. Suitable PHPG producing devices are known in the art and are disclosed in U.S. Pat. No. 8,168,122 issued May 1, 2012 and U.S. Pat. No. 8,685,329 issued Apr. 1, 2014. It will be appreciated, that the number and capacity of the PHPG producing devices necessary to achieve a concentration of at least 0.05 ppm DHP depends on the size of the clean room. In some aspects, an entire manufacturing facility is a clean room facility and the number of PHPG producing devices can be adjusted appropriately. In practice is has been determined that a single PHPG device can continuously maintain a space of about 425 m³ (about 15,000 ft³) at about 0.6 ppm. Smaller spaces of about 4.5 m³ (150 ft³) can be easily maintained at a level of about over 5.0 ppm with a single PHPG device.

As provided herein, suitable PHPG producing devices can comprise an enclosure, an air distribution mechanism, a source of ultraviolet light, and an air-permeable substrate structure having a catalyst on its surface wherein the airflow passes through the air-permeable substrate structure and directs the PHPG produced by the device out of the enclosure when the device is in operation. As used herein, an enclosure and air distribution system can be the ductwork, fans, filters and other parts of an HVAC system suitable for a clean room. In certain aspects, the PHPG device is provided after air filtration to maximize the production of PHPG and reduce losses of PHPG as the air moves through the system. In other aspects, a PHPG producing device may be a stand-alone device. In certain aspects, the PHPG generating device is capable of producing PHPG at a rate sufficient to establish a steady state concentration of PHPG of at least 0.005 ppm in a closed air volume of 10 cubic meters. In certain aspects, a PHPG generating device generates PHPG from water present in the ambient air. As used herein, the air distribution provides an airflow having a velocity from about 5 nanometers/second (nm/s) to 10,000 nm/s as measured at the surface of the air permeable substrate structure. As used herein, the substrate structure is an air permeable substrate structure having a catalyst on the surface configured to produce non-hydrated purified hydrogen peroxide gas when exposed to a light source and provided an airflow. As used herein, the air permeable substrate structure having a catalyst on its surface is between about 5 nanometers (nm) and about 750 nm in total thickness. As used herein, the catalyst on the surface of an air permeable substrate structure is a metal, a metal oxide, or mixtures thereof and may be tungsten oxide or a mixture of tungsten oxide with another metal or metal oxide catalyst.

As provided herein, PHPG generating devices that can be installed into existing HVAC systems (e.g., inline) or as stand alone units produce PHPG that is essentially free of ozone, plasma species, or organic species. As used herein, the term “substantially free of ozone” means an amount of ozone below about 0.015 ppm ozone. In an aspect, “substantially free of ozone” means that the amount of ozone produced by the device is below or near the level of detection (LOD) using conventional detection means. As used herein, substantially free of hydration means that the hydrogen peroxide gas is at least 99% free of water molecules bonded by electrostatic attraction and London Forces. Also as used herein, a PHPG that is substantially free of plasma species means hydrogen peroxide gas that is at least 99% free of hydroxide ion, hydroxide radical, hydronium ion, and hydrogen radical. As used herein, PHPG is essentially free of organic species comprises.

The present disclosure provides for and includes clean rooms having suitable HVAC systems that further comprise one or more PHPG generating devices sufficient to maintain the clean room at a concentration of 0.05 ppm DHP gas (e.g., inline PHPG generating devices). In certain aspects, the one or more PHPG generating devices are placed downstream of the various filters that comprise the HVAC system. In other aspects, the PHPG generating device can be placed upstream of one or more filters of the HVAC system. According to the present disclosure, the HVAC system may be a recirculated air system. Also included are HVAC systems that further comprise makeup air systems to replenish exhausted air and air lost due to leakage. In some aspects, the makeup air system includes one or more PHPG producing devices. In some aspects, the makeup air system comprises on or more filters selected from a 30% ASHRAE filter, a 60% ASHRAE filter, or a 95% ASHRAE filter. See American Society of Heating, Refrigerating and Air-Conditioning Engineers (ASHRAE) ANSI/AHRAE standard 52.2-2007, available on the internet, for example at www.airfilterplus.com/wp-content/uploads/2014/05/Koch-ASHRAE-book.pdf.

In aspects according to the present disclosure, the HVAC system includes one or more High Efficiency Particulate Air (HEPA) filtration system in accordance with Federal Standard 209. Also included in the present disclosure are HVAC systems that comprise at least one filter that is at least 99.97% efficient on 0.3 micron particles in accordance with Mil-F-51068 or IEST-RP-CC-001. As noted, the filtration systems can be placed upstream or downstream of an inline PHPG generating device.

The present disclosure also provides for, and includes, clean rooms having DHP gas having a variety of forms and differing approaches to meet the requirements, for example of ISO 14644. In certain aspects, the clean rooms may comprise a portable or modular clean room and include a PGPG generating device. In some aspects, clean rooms comprise turbulent flow HVAC systems to remove particles. In other aspects, clean rooms provide laminar flow of air to remove particles. As an added gas to the clean room air system, both laminar flow and turbulent flow systems can be prepared that comprise DHP gas at a concentration of at least 0.05 ppm.

The present disclosure provides for and includes clean rooms suitable for multiple purposes. In an aspect, the clean room is a pharmaceutical clean room. In another aspect, the clean room is a biopharmaceutical clean room. In yet another aspect, the clean room is a semiconductor manufacturing clean room. In other aspects, the clean room is a modular clean room.

In certain aspects according to the present disclosure, the clean room comprises reduced levels of airborne contaminants. Non-limiting examples of organic contaminants that are reduced according to the clean rooms and methods of the present disclosure are provided in Table 1 above. One of ordinary skill in the art would understand that the oxidative action of hydrogen peroxide is not specific. Accordingly, it is understood that few if any organic compounds would be resistant to oxidation and ultimately destruction. Oxidation is a process by which a carbon atom gains bonds to more electronegative elements, most commonly oxygen. In another aspect, oxidation reactions are those in which the central carbon of a functional group is transformed into a more highly oxidized form. A skilled artisan would understand that DHP gas oxidizes formaldehyde, carbon disulfide, carbohydrates, organophosphorus and nitrogen compounds, phenols, BTEX pesticides, plasticizers, chelants, and virtually any other organic requiring treatment. In one aspect, a carbon-carbon double bond of an alkene is susceptible to oxidation. In another aspect, a carbon-carbon triple bond of an alkyne is susceptible to oxidation.

In one aspect, DHP oxidizes an anthrogenic compound. In another aspect, DHP oxidizes cyanides, NOx/SOx, nitrites, hydrazine, carbonyl sulfide, or other reduced sulfur compounds. In another aspect, DHP oxidizes chlorofluorocarbons or chlorocarbons. In yet another aspect, DHP oxidizes methylene chloride. In one aspect, DHP oxidizes perchloroehtylene. In another aspect, DHP oxidizes styrene or limonene.

The present disclosure provides for and includes clean rooms having reduced levels of volatile organic compounds (VOC) and very volatile organic compounds (VVOC), such as formaldehyde. All volatile organic substances whose retention time in gas chromatography is between C₆ (hexane) and C₁₆ (hexadecane) are subsumed under volatile organic compounds. The very volatile organic compounds include, inter alia, also formic acid and formaldehyde. The expression aldehydes as used here comprises not only the volatile compounds but also all other aldehydes, in particular formaldehyde, unless stated otherwise.

In one aspect, with reference to organic molecules, oxidation is a process by which a carbon atom gains bonds to more electronegative elements, most commonly oxygen. In another aspect, oxidation reactions are those in which the central carbon of a functional group is transformed into a more highly oxidized form. A skilled artisan would understand that DHP gas oxidizes formaldehyde, carbon disulfide, carbohydrates, organophosphorus and nitrogen compounds, phenols, BTEX pesticides, plasticizers, chelants, and virtually any other organic requiring treatment. In one aspect, a carbon-carbon double bond of an alkene is susceptible to oxidation. In another aspect, a carbon-carbon triple bond of an alkyne is susceptible to oxidation.

The present disclosure also provides for reduced levels of airborne organic contaminants and organic contaminants that have settled on surfaces. In certain aspects, the clean rooms provide reduced levels of organic contaminants settled onto silicon wafers that are being manufactured in the clean room. As provided, the use of the clean room comprising DHP gas continues, thus providing for reductions of contamination of the products being manufactured.

The present disclosure provides for, and includes, clean rooms having internal surfaces comprising a variety of materials. Notably, DHP gas is compatible with building materials generally and is also compatible with the materials used to construct clean rooms. Specifically, clean room materials are generally characterized by their resistance to the formation of particles that can become airborne. Accordingly, the present disclosure provides for clean rooms comprising at least 0.05 ppm DHP gas having internal surfaces selected from the group consisting of phenolic plastic, glass reinforced plastic, steel, coated steel, aluminum, epoxy coated concrete block, drywall and vinyl, drywall having a high build finish, and other materials coated with a high build finish. In other aspects, the clean room internal surfaces are prepared form the materials as provided in Table 1. In an aspect, the high build finish includes polyurethanes, epoxy pain, baked enamel, or glossy paints. Suitable materials for the construction of clean rooms are known in the art. In contrast to current commercial clean rooms, clean rooms according to the present disclosure provide for the elimination of unwanted compounds that can be released by building materials generally, and certain materials used in the construction of clean rooms specifically.

The present disclosure provides for, and includes, clean rooms having a variety of air change rates. As noted above, the incorporation of DHP gas into the clean room is not restricted to whether the air in clean room is exchanged using laminar or turbulent flow. For turbulent flow clean rooms, air exchange is typically measured in terms of air changes per hour (ACH or ac/h). One of ordinary skill in the art would recognize that increased air exchange rates are associated with clean rooms having a lower classification (assuming no other change in the configuration of the filtration system). The present disclosure provides for the clean rooms comprising DHP gas at a level of at least 0.05 ppm having air change rates of at least 1 ACH. In an aspect, the air change rate is at least 5 ACH. In another aspect, the air change rate is at least 60 ACH. In a further aspect, the ACH is at least 150. In yet other aspects, the air change rate is at least 240 ACH. In some aspects, the air flow is at least 300 ACH. In some aspects, the air flow is at least 360 ACH. It is should be understood that the present disclosure provides for and includes even higher exchange rates per hour, achievable by incorporating additional PHGP generating devices.

The present disclosure provides for air exchange rates in clean rooms comprising DHP gas of between 5 and 48 ACH. In other aspects, the air exchange rate of clean rooms of the present disclosure is between 60 to 90 ACH. In some aspects, the air exchange rate is between 150 and 240 ACH. In additional aspects, the air exchange change rate is between 240 and 480 ACH. In another aspect, the air exchange change rate is between 300 and 540 ACH. In yet another aspect, the air exchange change rate is between 360 and 540 ACH.

The present disclosure provides for, and includes, clean rooms having a variety of laminar air flow velocities. In aspects according to the present disclosure, a clean room having at least 0.05 ppm DHP gas has an average airflow velocity of 0.005 m/s to 0.508 m/s. In certain aspects, the average airflow velocity may be greater than 0.508 m/s. In some aspects, the average airflow velocity is at least 0.005 m/s. In another aspect, the average airflow velocity is at least 0.051 m/s. In yet another aspect, the average airflow velocity is at least 0.127 m/s. In some aspects, the average airflow velocity is at least 0.203 m/s. In an aspect, the average airflow velocity is at least 0.254 m/s. In an additional aspect, the average airflow velocity is at least 0.305 m/s.

The present disclosure provides for, and includes, clean rooms having at least 0.05 ppm DHP gas and includes clean rooms having a range of air flow velocities. In an aspect, the laminar air flow velocity is between 0.005 and 0.041 m/s. In another aspect, the laminar air flow velocity is between 0.051 and 0.076 m/s. In another aspect, the laminar air flow velocity is between 0.127 and 0.203 m/s. In yet another aspect, the laminar air flow velocity is between 0.203 and 0.406 m/s. In another aspect, the laminar air flow velocity is between 0.254 and 0.457 m/s. In an aspect, the laminar air flow velocity is between 0.305 and 0.457 m/s. In a further aspect, the laminar air flow velocity is between 0.305 and 0.508 m/s. It will be understood that other flow rates are envisioned according to the present disclosure and that additional sources of PHPG can be incorporated into the system to provide suitable levels of DHP gas, up to 10 ppm and as provided at paragraphs [0046] and [0047].

The present disclosure provides for, and includes, clean rooms having at least 0.05 ppm DHP gas that have higher air pressures than adjacent non-clean room areas. A person of ordinary skill in the art would recognize that higher pressures can prevent the introduction of unwanted particles into the clean room. Not to be limited by theory, it is thought that when workers enter the clean room, the flow of air out of the clean room due to the difference in pressure acts to keep dust and particles from entering. In other aspects, the positive pressure can be provided to modular clean rooms to prevent the entrance of unwanted particles and microbes. The difference between the clean room and surrounding areas need only be sufficient to provide for a positive flow of air from the clean room. In aspects according to the present disclosure, the difference in pressure is at least 5 Pa. In an aspect, the difference pressure is at least 12 Pa. In an aspect, the pressure difference is at least 15 Pa. In an aspect, the pressure difference is at least 20 or 25 Pa. In other aspects, the pressure difference is at least 30 Pa. Also provided are pressure differences up to 50 Pa or even greater. In general, the pressure difference of a clean room comprising at least 0.05 ppm DHP gas and an adjacent non-clean room is between 5 and 50 Pa.

The present disclosure further includes and provides for, clean rooms further comprising an airlock or anteroom. As would be understood, such ancillary facilities are often included to minimize the introduction of contaminants. In certain aspects these associated facilities provide for lockers, changing rooms, airlocks, anterooms, and other functions. In certain aspects, these ancillary facilities, such as an airlock, a pass-through airlock, an anteroom, a changing room, interlock, or locker room further comprise DHP gas at a concentration of at least 0.05 ppm. Also included are ancillary facilities that have higher DHP gas levels as provided for example at paragraph [0046].

The present disclosure further includes and provides for, clean rooms that have a controlled environment. In certain aspects, the clean room is maintained at a temperature of between 20 to 22° C. In other aspects, the clean room is maintained at 18.9° C. Also included are cold clean rooms having a temperature of between 1 and 6° C.

Also included in the present disclosure are clean rooms having at least 0.05 ppm DHP gas and having a relative humidity of between 1 and 99%. In certain aspects, the relative humidity is between 30 and 60%. In an aspect, the humidity of the clean room air is preferably above about 1% relative humidity (RH). In other aspects, the humidity of the clean room air is at or above 5% RH. In further aspects, the humidity of the clean room air is at or above 10%. In some aspects, the relative humidity is between 35% and 40%. In other aspects, the humidity may be between about 5% and about 99% RH. In other aspects, the humidity of the clean room air may be between about 10% and about 99% RH. In certain aspects, the humidity of the clean room air is less than 80%. In an aspect, the humidity is between 10% and 80%. In yet other aspects, the relative humidity is between 30% and 60%. In another aspect, the humidity is between 35% and 40%. In some aspects, the humidity of the clean room air is between 56% and 59%.

As used herein, biocontainment environments are a subset of clean rooms that are designed to prevent materials, specifically living organisms such as bacteria and viruses, from exiting the room or facility. Accordingly, clean rooms designed to be biocontainment environments are engineered to operate under negative pressure wherein entry or exit from the biocontainment area results in air entering the clean room. As a result, biocontainment environments, while designed to remove particles and provide quality air like typical clean rooms, often are unable to achieve some of the very high levels of cleanliness associated for example with an ISO 14644 class 1 clean room. Like clean rooms however, regulatory authorities have established standards for biocontainment environments. The Centers for Disease Control and Prevention specifies rooms and facilities (e.g., multi room buildings) as biosafety level 1 (BSL-1), biosafety level 2 (BSL-2), biosafety level 3 (BSL-3), or biosafety level 4 (BSL-4). These standards are known to a person of ordinary skill and can be found on the internet at, for example, www.cdc.gov/biosafety/publications/bmb15/BMBL.pdf. The present disclosure provides for and includes clean rooms that comply with biocontainment environment specified by the Centers for Disease Control and Prevention as biosafety level 1 (BSL-1), biosafety level 2 (BSL-2), biosafety level 3 (BSL-3), or biosafety level 4 (BSL-4) and have at least 0.05 ppm DHP gas. Also as provided herein, biocontainment environments can have DHP gas at levels up to 10 ppm as discussed above. As will be understood, the addition of DHP gas to biocontainment environments provides an additional level of safety by reducing or eliminating the organisms or agents (bacteria, viruses, and toxins) the facility is designed to contain.

In an aspect according the present disclosure, the biocontainment environment may be a BSL-1 environment having at least 0.05 ppm DHP gas. In another aspect, the biocontainment environment may be a BSL-1 environment having between 0.05 and 10 ppm DHP gas. Additional levels of DHP gas suitable for BSL-1 environments are provided at paragraph [0046].

In an aspect according the present disclosure, the biocontainment environment may be a BSL-1 environment having at least 0.05 ppm DHP gas suitable for work on, but not limited to, Orthomyxoviridae, Alcaligenes faecalis, Aspergillus niger, Bacillus cereus, Bacillus megaterium, Bacillus subtilis, Clostridium sporogenes, Enterobacter aerogenes, Enterobacter cloacae, Escherichia coli, Micrococcus roseus, Micrococcus luteus, Mycobacterium smegmatis, Neisseria sicca, Neisseria subflava, Penicillium notatum, Rhizopus stolonifer, Rhodospirillum rubrum, Serratia marcescens, Staphylococcus epidermidis, Streptococcus bovis, or Streptococcus (Lactococcus) lactis.

In an aspect according the present disclosure, the biocontainment environment may be a BSL-2 environment having at least 0.05 ppm DHP gas. In another aspect, the biocontainment environment may be a BSL-2 environment having between 0.05 and 10 ppm DHP gas. Additional levels of DHP gas suitable for BSL-2 environments are provided at paragraph [0046].

In an aspect according the present disclosure, the biocontainment environment may be a BSL-2 environment having at least 0.05 ppm DHP gas suitable for work on, but not limited to, C. difficile, Chlamydia, hepatitis virus, non smallpox orthopoxvirudae, influenza, Lyme disease, Salmonella sp., mumps, measles, scrapie, methicillin-resistant Staphylococcus aureus (MRSA), or vancomycin-resistant Staphylococcus aureus (VRSA).

In an aspect according the present disclosure, the biocontainment environment may be a BSL-3 environment having at least 0.05 ppm DHP gas. In another aspect, the biocontainment environment may be a BSL-3 environment having between 0.05 and 10 ppm DHP gas. Additional levels of DHP gas suitable for BSL-3 environments are provided at paragraph [0046].

In an aspect according the present disclosure, the biocontainment environment may be a BSL-3 environment having at least 0.05 ppm DHP gas suitable for work on, but not limited to, Yersinia pestis, Francisella tularensis, Leishmania donovani, Mycobacterium tuberculosis, Chlamydia psittaci, Venezuelan equine encephalitis virus, Eastern equine encephalitis virus, SARS coronavirus, Coxiella burnetii, Rift Valley fever virus, Rickettsia rickettsia, Brucella sp., rabies virus, chikungunya, yellow fever virus, and West Nile virus.

In an aspect according the present disclosure, the biocontainment environment may be a BSL-4 environment having at least 0.05 ppm DHP gas. In another aspect, the biocontainment environment may be a BSL-4 environment having between 0.05 and 10 ppm DHP gas. Additional levels of DHP gas suitable for BSL-4 environments are provided at paragraph [0046].

In an aspect according the present disclosure, the biocontainment environment may be a BSL-4 environment having at least 0.05 ppm DHP gas suitable for work on, but not limited to, Arenaviridae, Filoviridae, Bunhaviridae, Flaviviridae, or Rhabdoviridae.

The present disclosure provides for and includes a method of preventing contamination of a clean room by microorganisms comprising providing a Dilute Hydrogen Peroxide (DHP) gas at a concentration of at least 0.05 parts per million (ppm) to said clean room. The antimicrobial activities of hydrogen peroxide are known generally and DHP gas provides a significant improvement over previous applications. In contrast to previous methods, DHP gas is non-toxic and suitable for use during occupation of the clean room to be treated. DHP gas does not settle and therefore can not contaminate surfaces of the clean room or manufactures being prepared in a clean room.

In aspects according to the present disclosure, the method of preventing contamination of a clean room by microorganisms includes providing DHP gas at the levels as recited at paragraph [0046] above. In certain aspects, the method includes providing DHP gas at up to 10 ppm. In certain aspects, the method includes providing DHP gas at least at between 0.05 and 10 ppm. In one aspect, the method includes providing DHP gas at least at 0.08 ppm. In another aspect, the method includes providing DHP gas at least at 1.0 ppm. In yet another aspect, the method includes providing DHP gas at least at 1.5 ppm. In one aspect, the method includes providing DHP gas at least at 2.0 ppm. In another aspect, the method includes providing DHP gas at least at 3.0 ppm. In one aspect, the method includes providing DHP gas at least at 5.0 ppm. In another aspect, the method includes providing DHP gas at least at 6.0 ppm. In one aspect, the concentration of DHP gas provided is less than 10 ppm. In one aspect, the concentration of DHP gas provided is less than 9.0 ppm. In another aspect, the concentration of DHP gas provided is less than 8.0 ppm. In an aspect, the concentration of DHP gas provided is less than 7.0 ppm. In another aspect, the concentration of DHP gas provided is between 0.05 ppm and 10.0 ppm. In yet another aspect, the concentration of DHP gas provided is between 0.05 ppm and 5.0 ppm. In one aspect, the concentration of DHP gas provided is between 0.08 ppm and 2.0 ppm. In yet another aspect, the concentration of DHP gas provided is between 1.0 ppm and 3.0 ppm. In one aspect, the concentration of DHP gas provided in a clean room of the present disclosure is between 1.0 ppm and 8.0 ppm, or between 5.0 ppm and 10.0 ppm. In other aspects, the concentration of DHP provided in a clean room cycles between higher and lower concentrations of DHP. By way of non-limiting example, the DHP may be provided at a higher concentration during the overnight hours and a lower concentration during the daytime hours.

In aspects according to the present disclosure, methods of preventing contamination of a clean room by microorganisms provides for reducing the numbers or eliminating microorganisms selected from the group consisting of comprise a virus, a viroid, a virus-like organism, a bacterium, a protozoa, an algae, an oomycete, a fungus, and a mold.

The present disclosure provides for, and includes, a method of reducing contamination of a clean room by microorganisms comprising providing a Dilute Hydrogen Peroxide (DHP) gas at a concentration of at least 0.05 parts per million (ppm) to said clean room. In aspects according to the present disclosure, the method of preventing contamination of a clean room by microorganisms includes providing DHP gas at the levels as recited at paragraphs [0046] and [0080] above.

In aspects according to the present disclosure, methods of reducing contamination of a clean room by microorganisms provides for reducing the numbers or eliminating microorganisms selected from the group consisting of comprise a virus, a viroid, a virus-like organism, a bacterium, a protozoa, an algae, an oomycete, a fungus, and a mold.

The present disclosure provides for, and includes, a method of eliminating contamination of a clean room by microorganisms comprising providing a Dilute Hydrogen Peroxide (DHP) gas at a concentration of at least 0.05 parts per million (ppm) to said clean room. In aspects according to the present disclosure, the method of eliminating contamination of a clean room by microorganisms includes providing DHP gas at the levels as recited at paragraphs [0046] and [0080] above.

In various aspects, the microorganisms may be selected from the group consisting of fungus, archaea, protest, protozoa, bacterium, bacterial spore, bacterial endospore, virus, viral vector, and combinations thereof. In other aspects, the microorganism may be selected from the group consisting of Naegleria fowleri, Coccidioides immitis, Bacillus anthraces, Haemophilus influenzae, Listeria monocytogenes, Neisseria meningitides, Staphylococcus aureus, Streptococcus pneumoniae, Streptococcus agalactiae, Pseudomonas aeruginosa, Yersinia pestis, Clostridium botulinum, Francisella tularensis, variola major, Nipah virus, Hanta virus, Pichinde virus, Crimean-Congo hemorrhagic fever virus, Ebola virus, Marburg virus, Lassa virus, Junin virus, human immunodeficiency virus (“HIV”), or SARS-associated coronavirus (“SARS-CoV”).

The methods of the present disclosure further provide to the reduction or elimination of microorganisms selected from the group consisting of S. Aureus, Alcaligenes Xylosoxidans, Candida Parapsilosis, Pseudomonas Aeruginosa, Enterobacter, Pseudomonas Putida, Flavobacterium Meningosepticum, Pseudomonas Picketti, Citrobacter, and Corynebacteria. The present disclosure further includes methods to reduce or eliminate C. difficile, Chlamydia, hepatitis virus, non smallpox orthopoxvirudae, influenza, Lyme disease, Salmonella sp., mumps, measles, scrapie, methicillin-resistant Staphylococcus aureus (MRSA), or vancomycin-resistant Staphylococcus aureus (VRSA). In additional aspects, the present disclosure provides for the reduction or elimination of Yersinia pestis, Francisella tularensis, Leishmania donovani, Mycobacterium tuberculosis, Chlamydia psittaci, Venezuelan equine encephalitis virus, Eastern equine encephalitis virus, SARS coronavirus, Coxiella burnetii, Rift Valley fever virus, Rickettsia rickettsia, Brucella sp., rabies virus, chikungunya, yellow fever virus, and West Nile virus.

The present disclosure provides for and includes methods to reduce or eliminate viruses. There are no known viruses of any type that are resistant to H₂O₂, whether provided as a gas, a liquid or a vapor. Importantly, providing an environment comprising DHP gas at a concentration of at least 0.05 ppm is effective against all types of virus that are exposed to the air. The methods of the present disclosure are effective against all classes of viruses including class I viruses comprising double stranded DNA (dsDNA) viruses including for example Adenoviruses, Herpesviruses, and Poxviruses; Class II viruses comprising single stranded DNA (ssDNA) viruses, for example Parvoviruses; Class III double stranded RNA (dsRNA) viruses including for example Reoviruses, Class IV viruses comprising plus strand single stranded ((+)ssRNA) viruses, for example Picornaviruses and Togaviruses; Class V viruses comprising minus strand single stranded RNA ((−)ssRNA) viruses, for example Orthomyxoviruses and Rhabdoviruses including Arenaviridae, Class VI viruses comprising single stranded RNA reverse transcribed (ssRNA-RT) viruses that have an RNA genome with DNA intermediate in life-cycle (e.g. Retroviruses); and Class VII viruses comprising double stranded DNA reverse transcribed (dsDNA-RT) viruses (e.g. Hepadnaviruses including Hepatitis viruses). It is expected that H₂O₂ gas is effective at inactivating and killing all viruses. Resistant viruses are not known.

The present disclosure provides for methods and compositions effective against all Class I viruses including but not limited to the group selected from Herpesviridae (including herpesviruses, Varicella Zoster virus), Adenoviridae, Asfarviridae (including African swine fever virus), Polyomaviridae (including Simian virus 40, JC virus, BK virus), and Poxviridae (including Cowpox virus, smallpox).

The present disclosure provides for methods and compositions effective against all Class III viruses including but not limited to Picobirnaviridae and Reoviridae (including Rotavirus).

The present disclosure provides for methods and compositions effective against all Class IV viruses including but not limited to the families selected from the group consisting of Coronaviridae (including Coronavirus, SARS), Picornaviridae (including Poliovirus, Rhinovirus (a common cold virus), Hepatitis A virus), Flaviviridae (including Yellow fever virus, West Nile virus, Hepatitis C virus, Dengue fever virus); Caliciviridae (including Norwalk virus also known as norovirus) and Togaviridae (including Rubella virus, Ross River virus, Sindbis virus, Chikungunya virus). The present disclosure provides for methods and compositions effective against norovirus.

The present disclosure provides for methods and compositions effective against all Class V viruses which includes nine virus families that comprise some of the most deadly viruses known. The methods of the present disclosure are effective at reducing or eliminating viruses of the families Arenaviridae, Bunyaviridae, Rhabdoviridae, Filoviridae, Paramyxoviridae.

The present disclosure provides for methods and compositions effective against all retroviruses of Class VI including but not limited to the group selected from Alpharetrovirus, Betaretrovirus, Gammaretrovirus, Deltaretrovirus; Epsilonretrovirus, and Lentivirus.

Family Bornaviridae (includes Boma disease virus); Filoviridae (includes Ebola virus, Marburg virus); Paramyxoviridae (includes Measles virus, Mumps virus, Nipah virus, Hendra virus, RSV and NDV); Rhabdoviridae (includes Rabies virus); Nyamiviridae (includes Nyavirus); Arenaviridae (includes Lassa virus); Bunyaviridae (includes Hantavirus, Crimean-Congo hemorrhagic fever); Ophioviridae (infects plants); and Orthomyxoviridae (includes Influenza viruses).

The present disclosure provides for methods and compositions effective against bacteria including gram positive and gram negative bacteria. The methods and compositions are effective against pathogenic bacteria including, but not limited to, Acinetobacter including Acinetobacter baumannii, Bacillus including Bacillus anthracis and Bacillus cereusl Bartonella including Bartonella henselae, and Bartonella quintana; Bordetella including Bordetella pertussis; Borrelia including Borrelia burgdorferi, Borrelia garinii, Borrelia afzelii, Borrelia recurrentis, and Borrelia duttonii; Brucella including Brucella abortus, Brucella canis, Brucella melitensis, and Brucella suis; Campylobacter including Campylobacter jejuni; Chlamydia and Chlamydophila including Chlamydia pneumoniae, Chlamydia trachomatis, and Chlamydophila psittaci, Clostridium including Clostridium botulinum, Clostridium difficile, Clostridium perfringens, and Clostridium tetani, Corynebacterium including Corynebacterium diphtheriae; Enterococcus including Enterococcus faecalis and Enterococcus faecium; Escherichia including Escherichia coli; Francisella including Francisella tularensis; Haemophilus including Haemophilus influenzae; Helicobacter including Helicobacter pylori, Legionella including Legionella pneumophila; Leptospira including Leptospira interrogans, Leptospira santarosai, Leptospira weilii, and Leptospira noguchii; Listeria including Listeria monocytogenes; Moraxella including M. catarrhalis; Mycobacterium including Mycobacterium leprae, Mycobacterium tuberculosis, and Mycobacterium ulcerans; Mycoplasma including Mycoplasma pneumoniae; Neisseria including Neisseria gonorrhoeae, and Neisseria meningitidis; Pseudomonas including Pseudomonas aeruginosa; Rickettsia including Rickettsia rickettsia; Salmonella including Salmonella typhi, and Salmonella typhimurium; Shigella including Shigella sonnei; Staphylococcus including Staphylococcus aureus, Staphylococcus epidermidis, and Staphylococcus saprophyticus; Streptococcus including Streptococcus agalactiae, Streptococcus pneumoniae, and Streptococcus pyogenes; Treponema including Treponema pallidum; Vibrio including Vibrio cholerae; Yersinia including Yersinia pestis, Yersinia enterocolitica, and Yersinia pseudotuberculosis.

The present disclosure provides for methods and compositions effective against antibiotic resistant bacteria, including but not limited to, Methicillin Resistant Staphylococcus Aureus (MRSA), Vancomycin Resistant Enterococcus Faecalis (VRE)

The present disclosure provides for methods and compositions effective against fungal and mold pathogens, including without limitation, Aspergillus spp., Candida albicans, Sclerotinia or Pneumocystis spp. In another aspect, the fungi is from the genus Mucoraceae. In other aspects, the present disclosure provides for methods and compositions effective against a fungus selected from the group consisting of Histoplasma capsulatum, blastomyces, Cryptococcus neoformans, Pneumocystis jiroveci, Coccidioides immitis, Blastomyces dermatitides, Pneumocystis jirovecii, Sporothrix schenckii, Cryptococcus neoformans, Aspergillus fumigatus, and Candida albicans.

The present disclosure provides for, and includes, a method of reducing organic compounds in clean room comprising providing a Dilute Hydrogen Peroxide (DHP) gas at a concentration of at least 0.05 parts per million (ppm) to said clean room. In aspects according to the present disclosure, the method of reducing organic compounds includes providing DHP gas at the levels as recited at paragraphs [0046] and [0080] above.

The present disclosure provides for, and includes a method of reducing the levels of volatile organic species in a clean room comprising providing a Dilute Hydrogen Peroxide (DHP) gas at a concentration of at least 0.05 parts per million (ppm) to said clean room. In aspects according to the present disclosure, the method of reducing organic compounds includes providing DHP gas at the levels as recited at paragraphs [0046] and [0080] above.

In certain aspects, a method of reducing the levels of volatile organic species in a clean room includes reductions in one or more volatile organic species are selected from the group consisting of bis(2-ethylhexyl) benzene-1,2-dicarboxylate (DOP), triethylphosphate (TEP), butylated hydroxytoluene (BHT), texanol isobutyrate (TXIB), tributyl phosphate (TBP), dibutyl phosphate (DBP).

The present disclosure provides for, and includes a method of providing Dilute Hydrogen Peroxide (DHP) gas at a concentration of at least 0.05 parts per million (ppm) to a clean room comprising installing a PHPG generating device. In aspects according to the present disclosure, the PHPG generating device is a device as provided above at paragraph [0050]. In aspects according to the present disclosure, a PHPG generating device comprises an air-permeable substrate structure having a catalyst on its surface, a source of light; and wherein air flows through said air-permeable substrate structure and the device produces PHPG and directs it away from said air-permeable substrate structure. In an aspect, the light source of the PHPG generating device is a UV light. In certain aspects, the UV light of a PHPG generating device does not include wavelengths of light below 187 nm. In certain aspects, the PHPG generating device includes a fan to provide air flow through the air-permeable substrate structure. In other aspects, the HVAC system provides the air flow.

Suitable PHPG generating devices of the present disclosure produce DHP gas that is substantially free of ozone, plasma species, or organic species. Suitable PHPG generating devices do not prepare DHP gas from vaporized hydrogen peroxide liquid. Accordingly, the DHP gas of the method is non-hydrated. In an aspect, the PHPG generating device is included as a component of a heating ventilation and air conditioning (HVAC) system. In other aspects, the PHPG generating device may be a stand-alone device. In aspects according to the present disclosure, the PHPG generating device of the method generates DHP gas from humid ambient air.

The present disclosure provides for, and includes a method of reducing organic species adsorption-induced contamination during silicon wafer production comprising providing Dilute Hydrogen Peroxide (DHP) gas at a concentration of at least 0.05 parts per million (ppm) to a silicon wafer production facility clean room. In aspects according to the present disclosure, method of reducing organic species adsorption-induced contamination during silicon wafer production includes providing DHP gas at the levels as recited at paragraphs [0046] and [0080] above.

As provided herein, a method of reducing organic species adsorption-induced contamination during silicon wafer production includes the reduction of organic species selected from the group consisting of stearic acid, butylated hydroxy toluene, siloxane, 4-dodecylbenzenesulfonic acid, n-pentadecane, bis(2-ethylhexyl) benzene-1,2-dicarboxylate (DOP), 3,4-dibutylphthalic acid (DBP), diethylphthalate (DEP), tris(2-chloroethyl) phosphate (TCEP), triphenyl phosphate (TPP), triethyl phosphate (TEP), hexanedioate (DOA), 2,2-dibutylhexanedioic acid (DBA), and 2,6-ditert-butyl-4-methylphenol (BHT). In an aspect, the organic species adsorption-induced contamination is reduced by at least 10%.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

### EXAMPLES

### Example 1: Laboratory Testing of DHP for the Control of Geobacillus subtillus Spores

The effects of DHP gas on Geobacillus subtillus spores is performed to determine the efficacy on killing the spores using the indirect dispersion of DHP gas in a space. In these experiments, the mortality rates in G. subtillus spores is assayed using filter strip impregnated with G. subtillus spores which are subjected to DHP gas. The test strips provide a visual readout following exposure to DHP for a specific period of time. The G. subtillus impregnated test strips are first exposed to DHP and them dipped in a tryptic soy broth solution and placed on a dry bath for a 24-hour incubation period. Following the incubation period, each test strip is analyzed to determine the presence of any viable bacteria. A change in color or the presence turbidity prior to the expiration of the 24-hour incubation period indicates that viable spores remain following exposure to DHP. Conversely, an absence of a change in color or turbidity prior to the expiration of the 24-hour incubation period indicates the eradication of the G. subtillus spores. The results are presented in Table 2 below.

TABLE 2
Effect of DHP on Geobacillus subtillus spores in Laboratory Tests

| | | Biological | Color Change |
|---|---|---|---|
| | Exposure to DHP | Change/Time of | within 24 hour |
| Spore Strip | (hours) | Change (hours) | incubation? |
| Log3 | 40 | Heavy turbidity | +Light orange |
| Log3 | 42 | x | + |
| Log3 | 45.5 | x | + |
| Log3 | 47.75 | Less turbidity | +Dark orange |
| Log3 | 64.5 | x | + |
| Log3 | 70 | x | + |
| Log3 | 60.2 | x | + |
| Log3 | 64.2 | x | + |
| Log3 | 67.5 | x | + |
| Log3 | 85.1 | x | + |
| Log3 | 89 | x | + |
| Log3 | 100 | 16 | + |
| Log3 | 60.2 | Heavy turbidity | + |
| Log3 | 64.2 | x | + |
| Log3 | 67.5 | Almost no turbidity | + |
| Log3 | 85.1 | x | + |
| Log3 | 89 | x | + |
| Log3 | 100 | 22-24 | + |
| Log4 | 121.4 | almost no turbidity | − |
| | | 8-15 | |
| Log4 | 144 | almost no | − |
| | | turbidity 17 | |
| Log4 | 168 | | − |
| Log4 | 192 | 15 | − |
| Log4 | 223.5 | 14 | − |
| Log4 | 288 | no turbidity | darker orange |
| | | 17 | |
| Log4 | 121.4 | almost no | − |
| | | turbidity 15 | |
| Log4 | 144 | [00107] almost o | − |
| | | turbidity 17 h | |
| Log4 | 168 | | − |
| Log4 | 192 | 15 | − |
| Log4 | 223.5 | no turbidity | light orange |
| | | 22 | |
| Log2 | 288 | no turbidity | very light orange |
| | | 17 | |
| Log2 | 72 | turbidity | light yellow |
| | | 15 | |
| Log2 | 144 | 22 | very dark orange |
| | | | almost light |
| | | | purple |
| Log3 | 144 | no turbidity | very light orange |
| | | 16.5 | |
| Log4 | 144 | no turbidity | very light orange |
| | | 16.5 | |
| Log2 | 166.5 | | may have |
| | | | changed prior to |
| | | | 24 hours but still |
| | | | dark |
| Log3 | 166.5 | | − |
| Log4 | 166.5 | | − |
| Log2 | 216 | no turbidity | very dark orange |
| | | 22 | |
| Log3 | 216 | no turbidity | very dark orange |
| | | 22 | |
| Log4 | 216 | no turbidity | very dark orange |
| | | 22 | |

### Example 2: Application of Modular Clean Rooms to Bottling Facilities

Soft drink bottling facilities (e.g., canning) require a high level of cleanliness to prevent the contamination of the products during production. To achieve this, the bottling machinery is equipped with air filtration systems that maintain a sterile environment. In practice, a filter equipped bottling line can bottle about 3×10⁶ cans before the filter requires replacing. The filters are very expensive and contribute a significant amount to the overall production costs of the finished product. Filters are changed at regular intervals when the filtered air quality drops below specified requirements.

To increase the life of the filters, a custom built 7×4×4 foot modular clean room is constructed around a bottling machine in a production facility and equipped with a PHPG generative device. The custom built modular clean room encloses the canning machine leaving a 3 inch gap at the base. Thus the modular clean room enclosing the canning maching operates at a higher air pressure than the surrounding area as provided above at paragraph [0066]. The modular clean room is equipped with a ventilation system, separate from the HVAC system of the facility, providing filtered, humid (˜60%) air and is further equipped with a PHPG generating device as described in International Patent Publication No. WO 2015/171633. Using the PHPG device, the modular clean room enclosing the bottling filter and equipment is continuously maintained at a level of 5.0 ppm DHP gas. When operated in the presence of PHPG, the filter continues to maintain the required levels of filtration for at least six weeks providing a production line equivalent of 12×10⁶ cans of product. The application of DHP technology results in at least a four fold increase in the life span of the filter resulting in significant cost savings.

1. A clean room comprising dilute hydrogen peroxide (DHP) gas at a concentration of at least 0.05 parts per million (ppm) up to 10.0 ppm and 0.015 parts per million (ppm) ozone or less.

2. (canceled)

3. The clean room of claim 1, wherein said clean room is an ISO 14644 class 1 clean room, an ISO 14644 class 2 clean room, an ISO 14644 class 3 clean room, an ISO 14644 class 4 clean room, an ISO 14644 class 5 clean room, an ISO 14644 class 6 clean room, an ISO 14644 class 7 clean room, or an ISO 14644 class 8 clean room.

4. The clean room of claim 1, wherein said clean room is a BS 5295 class 1 clean room, a BS 5295 class 2 clean room, a BS 5295 class 3 clean room, or a BS 5295 class 4 clean room.

5. The clean room of claim 1, wherein said clean room is an EU GMP grade A clean room, an EU GMP grade B clean room, an EU GMP grade C clean room, an EU GMP grade D clean room.

6. The clean room of claim 1, wherein said clean room is a modular clean room.

7-15. (canceled)

16. The clean room of claim 1, wherein said makeup air system comprises one or more filters selected from a 30% ASHRAE filter, a 60% ASHRAE filter, or a 95% ASHRAE filter.

17-19. (canceled)

20. The clean room of claim 1, wherein said clean room is a turbulent flow clean room.

21. The clean room of claim 1, wherein said clean room is a laminar flow clean room.

22. The clean room of claim 1, wherein said clean room comprising DHP gas is safe for continuous human occupation according to the Occupational Safety and Health Administration (OSHA), National Institute for Occupational Safety and Health (NIOSH), or American Conference of Industrial Hygienists (ACGIH) standards.

23. The clean room of claim 1, wherein said clean room is a pharmaceutical clean room.

24. The clean room of claim 1, wherein said clean room is a biopharmaceutical clean room.

25. The clean room of claim 1, wherein said clean room is a clean room for semiconductor manufacture.

26. The clean room of claim 1, wherein said clean room has reduced levels of airborne molecular contaminants.

27. (canceled)

28. The clean room of claim 1, wherein the air change rate is between 1 and 360 air changes per hour (ACH).

29. The clean room of claim 21, wherein the average air velocity is between 0.005 m/s to 0.508 m/s.

30-38. (canceled)

39. The clean room of claim 1, wherein said clean room comprises a biocontainment environment specified by the Centers for Disease Control and Prevention as biosafety level 1 (BSL-1), biosafety level 2 (BSL-2), biosafety level 3 (BSL-3), or biosafety level 4 (BSL-4).

40-51. (canceled)

52. A method of providing DHP gas at a concentration of at least 0.05 parts per million (ppm) to said clean room comprising installing a PHPG generating device comprising an air-permeable substrate structure having a catalyst on its surface, a source of light; and wherein air flows through said air-permeable substrate structure and the device produces PHPG and directs it away from said air-permeable substrate structure.

53. The method of claim 52, wherein said DHP gas produced by said PHPG generating device is free of hydration, ozone, plasma species, and organic species.

54-55. (canceled)

56. The method of claim 52, wherein said DHP gas is not prepared from vaporized hydrogen peroxide liquid.

57-61. (canceled)

62. A method of reducing organic species adsorption-induced contamination during silicon wafer production comprising providing DHP gas at a concentration of at least 0.05 parts per million (ppm) to a silicon wafer production facility clean room.

63. The method of claim 62, wherein said organic species are selected from the group consisting of stearic acid, butylated hydroxy toluene, siloxane, 4-dodecylbenzenesulfonic acid, n-pentadecane, bis(2-ethylhexyl) benzene-1,2-dicarboxylate (DOP), 3,4-dibutylphthalic acid (DBP), diethylphthalate (DEP), tris(2-chloroethyl) phosphate (TCEP), triphenyl phosphate (TPP), triethyl phosphate (TEP), hexanedioate (DOA), 2,2-dibutylhexanedioic acid (DBA), and 2,6-ditert-butyl-4-methylphenol (BHT).

64. (canceled)
